(19) United States
(12) Patent Application Publication
(10) Pub. No.: US 2006/0045914 A1
(43) Pub. Date: Mar. 2, 2006

(54) Matrix composition for stable microemulsions

(75) Inventors: Kolazi S. Narayanan, Wayne, NJ (US); Domingo Jon, New York, NY (US); Jayanti Patel, Elmwood Park, NJ (US)

Correspondence Address: Attn: William J. Davis, Esq.;INTERNATIONAL SPECIALTY PRODUCTS, Legal Department, Building No. 8, 1361 Alps Road, Wayne, NJ, 07470, US

(73) Assignee: ISP INVESTMENTS INC.

(21) Appl. No.: 10/926,510

(22) Filed: Aug. 26, 2004

(51) Int. Cl. A61K 9/14

(52) U.S. Cl. 424/484

(57) ABSTRACT

A matrix composition for making a stable microemulsion for an active ingredient includes a C₈-C₁₈ N-alkyl pyrrolidone with little or no N-methyl pyrrolidone, and a water-insoluble organic solvent soluble therein, preferably more hydrophilic than said C₈-C₁₈ alkyl pyrrolidone and with low water solubility.

### CROSS-REFERENCE TO RELATED PATENTS

The art has described microemulsion concentrates for an active ingredient. See Narayanan U.S. Pat. No. 6,045,816—issued Apr. 4, 2000 “Water-Based Microemulsion of a Pyrethroid” (Microflex®); U.S. Pat. No. 6,187,715—issued Feb. 13, 2001 “Water Based Microemulsions of a Lower Alkyl Ester of Quinoxalinyl Herbicide”; Narayanan U.S. Pat. No. 6,251,416—issued Jun. 26, 2001 “Water-Based Microemulsion of a Pyrethroid” (Microflex®)—covers the concentrates; Narayanan U.S. Pat. No. 6,541,516—issued Apr. 1, 2003 “Water Miscible Emulsions of Pyrethroid Insecticides or Triazole Fungicides”: extension of Microflex using hydrophobic solvents for better stability on dilution.

### BACKGROUND OF THE INVENTION

1. Field of the Invention

This invention relates to a matrix composition for microemulsion concentrates containing a desired active ingredient or mixtures thereof, and, more particularly, to a matrix with little or no N-methylpyrrolidone including a C₈-C₁₈ N alkyl pyrrolidone and an organic solvent which is soluble therein, and preferably more hydrophilic than the C₈-C₁₈ alkyl pyrrolidone and with poor solubility in water.

2. Description of the Prior Art

However, the microemulsion concentrates of the above related patents require a large amount of N-alkyl pyrrolidones including N-methylpyrrolidone for increased loading of the active ingredients. N-methylpyrrolidone is listed under California Proposition 65 with certain labeling restrictions. Furthermore, the use of the hydrophobic solvents disclosed in U.S. Pat. Nos. 6,541,516 and 6,187,715 limits the type of active ingredients and the loading depending on the solubility of the active ingredients in the hydrophobic solvent chosen.

Accordingly, it is an object of this invention to provide a matrix composition for new and improved stable microemulsion concentrates which does not require N-methylpyrrolidone in concentrates containing a high loading of different active ingredients.

### SUMMARY OF THE INVENTION

A matrix composition for making a stable microemulsion concentrate of an active ingredient consisting essentially of, by wt.,
(a) 5-30% C₈-C₁₈ N alkyl pyrrolidone,
(b) 5-60% an organic solvent which is soluble in (a), selected from the group consisting of alcohols, ethers, esters, ketones, aldehydes, aliphatic and aromatic, and cyclic hydrocarbons, naturally occurring flavoring agents, vegetable oils, flavoring agents, fragrances, and monomers, e.g. propylene carbonate, propylene glycol, reduced vinyl pyrrolidone dimer, gamma-butyrolactone, N,N dimethyl imidazolidone, cyclohexanone and methyl ethyl ketone, benzophenone, benzyl benzoate esters of long chain carboxylic acid with greater than 4 carbon atoms or esters with an alkyl group (from the alcohol segment has more than 4 carbon atoms, alcohols having greater than six carbons, preferably which are more hydrophilic than (a), or hydrocarbon solvents for those active ingredients having high solubility, preferably more hydrophilic than (a) and with low water solubility,
(c) 30-70% of a non-ionic emulsifier, e.g. castor oil ethoxylate, which are alkoxylated oils with an HLB between 6-18, preferably 8-16,
(d) 1-15% of an EO/PO/EO copolymer, and optional ingredients includes
(e) 0-5% of a branched alkyl ethoxylated phosphoric acid to provide the desired pH on dilution in water depending on the active ingredient.

The matrix is made into a stable microemulsifiable concentrate when it includes
(f) 1-30%, of an active ingredient, or mixtures of active ingredients, preferably 3-20%.

Upon dilution with water, the concentrate forms a stable microemulsion composition of the invention.

In this invention, the active ingredient then can be delivered to a desired site by applying the stable microemulsion composition thereto.

Suitably, the active ingredient (f) is a biocide, or agricultural chemical, e.g. a fungicide, insecticide or herbicide.

Alternatively, the active ingredient can be an organic solvent, a fragrance, a personal care composition, e.g. a shampoo, a water-insoluble monomer, e.g. methyl methacrylate, styrene, an alkyl maleate or cinnamate, an olefinic hydrocarbon, e.g. an ester or amide or flavoring agent.

### EXAMPLES

The compositions shown below were prepared by weighing in appropriate quantities of the ingredients to make up 100 g samples in a 2-ounce stoppered bottles. The contents were dissolved using a rotary shaker over a period of 16 hours. All compositions were homogeneous at room temperature.

Stability evaluation of the samples were carried out as previously described in U.S. Pat. No. 6,045,816. The stability of the concentrates and on dilution are shown below.

### Example 1

### Preparation of Matrix of Invention

A. 100 g matrix composition was prepared by dissolving the following ingredients in a 2-ounce stoppered bottle. 12.5 g N octylpyrrolidone, 74.5 g castor oil ethoxylate (30EO), 11.0 g EO/PO (PEG oil<31) copolymer, and 2.0 g branched ethoxylated phosphate ester (9-10 EO).

B. 78.57 g of A. was mixed with 21.43 g of reduced vinyl pyrrolidone dimer (RVPD), 1,3-bis-(pyrrolidon-1-yl)butan as solvent to produce a homogeneous solution of the desired matrix composition.

### Example 2

Example 1 was repeated using propylene carbonate in place of RVPD.

### Example 3

90 g of Example 1A was mixed with 10 g of propylene carbonate.

### Example 4

20 g of Permetrin was dissolved in 80 g of the matrix of Example 1.

### Example 5

20 g of Permetrin was dissolved in 80 g of the matrix of Example 2.

Stability of Concentrates

All concentrate compositions shown herein were clear, homogeneous solutions at ambient conditions and at 50° C. and at 0° C., when stored for three weeks. All samples passed the standard freeze thaw cycle three times of alternate storage at 50° C. and 0° C. through room temperature for 24 hours at each temperature without any separation.

Stability on Dilution

Each of the concentrates shown herein were diluted with deionized water as well as 1000 ppm WHO hard water at the rates: 1/10, 1/100, and 1/1000 and any separation was noted by visual observation as a function of time during storage for 100 days at room temperature (22° C.-25° C.) and at 4° C. The results are summarized below:

All samples at the above dilutions: 1/10, 1/100, and 1/1000 when stored at ambient temperature 22-25° C. were clear, monitored for 100 days of storage.

TABLE 1

| Sample | Example 4 | Example 5 |
|---|---|---|
| Room Temp | | |
| Day 1 | | |
| Concentrate | clear | clear |
| 1/10 | clear | clear |
| 1/100 | clear | clear |
| 1/1000 | clear | clear |
| Day 15 | | |
| Concentrate | clear | clear |
| 1/10 | clear | clear |
| 1/100 | clear | clear |
| 1/1000 | clear | clear |
| Day 30 | | |
| Concentrate | clear | clear |
| 1/10 | clear | clear |
| 1/100 | clear | clear |
| 1/1000 | clear | clear |
| Day 100 | | |
| Concentrate | clear | clear |
| 1/10 | clear | clear |
| 1/100 | clear | clear |
| 1/1000 | clear | clear |

TABLE 2

| Sample | Example 4 | Example 5 |
|---|---|---|
| Temp = 4° C. | | |
| Day 1 | | |
| Concentrate | clear | clear |
| 1/10 | clear | clear |
| 1/100 | clear | clear |
| 1/1000 | clear | clear |
| Day 15 | | |
| Concentrate | clear | clear |
| 1/10 | clear | clear |
| 1/100 | clear | clear |
| 1/1000 | clear | clear |
| Day 30 | | |
| Concentrate | clear | clear |
| 1/10 | clear | clear |
| 1/100 | clear | clear |
| 1/1000 | clear | clear |
| Day 100 | | |
| Concentrate | clear | clear |
| 1/10 | clear | clear |
| 1/100 | clear | clear |
| 1/1000 | clear | clear |

TABLE 3

| Sample | Example 4 | Example 5 |
|---|---|---|
| Temp = 45° C. | | |
| Day 1 | | |
| Concentrate | clear | clear |
| 1/10 | clear | clear |
| 1/100 | clear | clear |
| 1/1000 | clear | clear |
| Day 15 | | |
| Concentrate | clear | clear |
| 1/10 | clear | ppts |
| 1/100 | clear | clear |
| 1/1000 | clear | clear |
| Day 30 | | |
| Concentrate | clear | clear |
| 1/10 | clear | ppts |
| 1/100 | clear | clear |
| 1/1000 | clear | clear |
| Day 100 | | |
| Concentrate | clear | clear |
| 1/10 | clear | ppts |
| 1/100 | clear | clear |
| 1/1000 | clear | clear |

### Example 6

Example 1B was repeated except 28 g of hexyl acetate was dissolved in 72 g of Matrix 1A. Diluted solutions of 1/10, 1/100 and 1/1000 were clear.

### Example 7

Example 6 was repeated except 50 g of hexyl acetate was used. Only diluted solutions of 1/100 and 1/1000 were clear.

### Example 8

Example 7 was repeated except 10 g of ethyl caprylate was dissolved in 90 g of Matrix 1A was used. Diluted solutions of 1/10, 1/100 and 1/1000 were clear.

### Example 9

Example 8 was repeated except 30 g of ethyl caprylate was used. Only diluted solution of 1/100 and 1/1000 was clear.

### Example 10

Example 9 was repeated except 30 g of ethyl caprylate was dissolved in 60 g of Composition of Example 1A. Diluted solutions of 1/100 and 1/1000 were clear.

### Example 11

Example 10 was repeated except 40 g of ethyl caprylate was used. Only diluted solution of 1/1000 was clear.

### Example 12

Example 11 was repeated except 10 g of citral was dissolved in 90 g of Composition of Example 1A. Diluted solutions of 1/10, 1/100 and 1/1000 were clear.

### Example 13

Example 12 was repeated except 40 g of citral was used. Only diluted solutions of 1/100 and 1/1000 were clear.

### Example 14

Example 13 was repeated except 10 g mild orange oil was dissolved in 90 g of Composition of Example 1A. Diluted solutions of 1/10, 1/100 and 1/1000 were clear.

### Example 15

Example 14 was repeated except 40 g of mild orange oil was used. Only diluted solutions of 1/100 and 1/1000 were clear.

### Example 16

Example 15 was repeated except 10 g valencia orange oil was dissolved in 90 g of Composition shown in Example 1A. Diluted solutions of 1/10, 1/100 and 1/1000 were clear.

### Example 17

Example 16 was repeated except 40 g of valencia orange oil was used. Only diluted solutions of 1/100 and 1/1000 were clear.

### Example 18

Example 17 was repeated except 20 g hexyl acetate was dissolved in 80 g of Composition of Example 1A. Only diluted solutions of 1/100 and 1/1000 were clear.

### Example 19

20 g Dipentene was dissolved in 80 g of Composition of Example 1A. Only diluted solutions of 1/100 and 1/1000 were clear.

### Example 20

Example 19 was repeated except 20 g benzyl benzoate was dissolved in 80 g of Composition of Example 1A. Diluted solutions of 1/10, 1/100 and 1/1000 were clear.

### Example 21

Example 20 was repeated except 30 g of benzyl benzoate was used. Only diluted solution 1/1000 was clear.

### Example 22

Example 21 was repeated except 10 g methyl methacrylate was dissolved in 90 g of Composition of Example 1A. Diluted solutions of 1/10, 1/100 and 1/1000 were clear.

### Example 23

Example 22 was repeated except 50 g of methyl methacrylate was used. Only diluted solution 1/100 and 1/1000 were clear.

### Example 24

10 g benzyl benzoate and 10 g of permethrin were dissolved in 80 g of Composition of Example 1A. This concentrate was diluted to 1/10, 1/100 and 1/1000. All dilutions were clear.

### Example 25

15 g benzyl benzoate and 15 g of permethrin were dissolved in 70 g of Composition of Example 1A. This concentrate was diluted to 1/10, 1/100 and 1/1000. All dilutions were clear.

### Example 26

Example 25 was repeated using 20 g benzyl benzoate and 20 g of permethrin were dissolved in 60 g of Composition of Example 1A. This concentrate was diluted to 1/100 and 1/1000 and were found to be clear.

All concentrates and clear dilutions remained clear for a period of 4 weeks.

What is claimed is:

1. A matrix composition consisting essentially of, by wt.,
(a) 5-30% C₈-C₁₈ alkyl pyrrolidone,
(b) 5-60% an organic solvent which is soluble in (a),
(c) 30-70% of a non-ionic emulsifier,
(d) 1-15% of an EO/PO/EO copolymer,
(e) 0-5% of a branched alkyl ethoxylated phosphoric acid.

2. A matrix composition according to claim 1 wherein
(b) is a solvent selected from the group consisting of alcohols, ethers, esters, ketones, aldehydes, aliphatic and aromatic, and cyclic hydrocarbons, naturally occurring flavoring agents, vegetable oils, flavoring agents, fragrances and monomers.

3. A matrix composition according to claim 2 wherein (b) is propylene carbonate, propylene glycol, reduced vinyl pyrrolidone dimer, gamma-butyrolactone, N,N dimethyl imidazolidone, cyclohexanone, methyl ethyl ketone, benzophenone, a benzyl benzoate, ester of a long chain carboxylic acid with greater than 4 carbon atoms or esters with an alkyl group wherein the alcohol segment has more than 4 carbon atoms or an alcohol having greater than 6 carbons.

4. A matrix composition according to claim 2 wherein (b) is more hydrophilic than (a).

5. A matrix composition according to claim 1 which includes
(f) 1-30% of an active ingredient.

6. A stable microemulsion comprising the stable microemulsion concentrate of claim 1, and water of dilution.

7. A method of delivering an active ingredient to a desired site which comprises applying the stable microemulsion of claim 6 thereto.

8. A stable microemulsion concentrate according to claim 5 wherein said active ingredient is a biocide, or an agricultural chemical selected from a fungicide, insecticide and herbicide.

9. A method according to claim 7 which delivers a medicated hair treating chemical, a fragrance selected from an orange oil, peppermint oil, rose oil, sandalwood oil or jasmine oil, at a concentration of 30-40% which on dilution to 1/100 to 1/1000 produces clear systems, or a water insoluble monomer selected from methyl methacrylate, styrene, alkyl maleate, alkyl cinnamate or an olefinic hydrocarbon or ester or amide.
